(19) United States
(12) Patent Application Publication
(10) Pub. No.: US 2012/0276641 A1
(43) Pub. Date: Nov. 1, 2012

(54) MICROFLUIDIC DEVICE PROVIDING DEGASSING DRIVEN FLUID FLOW

(75) Inventors: Ivan Dimov, Puerto Montt (CL); Jose L. Garcia-Cordero, Calgary (CA); Lourdes Basabe-Desmonts, Cartagena (ES)

(73) Assignee: DUBLIN CITY UNIVERSITY, Dublin (IE)

(21) Appl. No.: 13/504,108

(22) PCT Filed: Oct. 28, 2010
(86) PCT No.: PCT/EP10/66402

§ 371 Date: Jul. 16, 2012

(30) Foreign Application Priority Data
Oct. 30, 2009 (GB) 0919053.9

(51) Int. Cl. G01N 33/48; B23P 17/04; B01L 3/00

(52) U.S. Cl. 436/63; 422/63; 422/504; 29/890.09

(57) ABSTRACT

A device for blood-plasma separation and plasma-based blood analysis is described. The device uses blood samples smaller than 5 μL, (directly from the finger) and flow is achieved with a degassing-driven flow technique that causes blood to flow spontaneously into air-filled dead-end channels without external pumping mechanisms.

### FIELD OF THE INVENTION

The present invention relates to microfluidic devices and more particularly to microfluidic devices for fluid separation and/or combination and analysis purposes. The invention more particularly relates to a device for blood-plasma separation and plasma-based blood analysis that provides for fluid flow without external pumping or driving elements.

### BACKGROUND

Microfluidic devices are well known in the art and typically comprise a plurality of individual cavities or fluid channels defined within a substrate and through which a fluid may be stored or flow. The dimensions of the individual cavities or channels are typically of the order of a human hair. Usually the length of such channels is much greater than their width. Such kinds of channels, their geometrical variations and their networks are used in the microfluidic devices for various purposes such as DNA sequencing, separation by electrophoresis, cell sorting and culturing, biomolecular analysis, biological and chemical synthesis. Within the art, the development of microfluidic devices not only introduced possible miniaturization of the existing analytical technologies but also new opportunities to conduct novel experiments in non conventional formats for mining information otherwise difficult to obtain.

Within the context of fluid analysis such microfluidic devices offer the opportunity to analyse constituents of the fluids within very small sample volumes. This is particularly advantageous in the context of living fluids such as for example blood, where it is desired to provide an assay or analysis based on a small available sample volume.

Blood is a treasure of information about the condition of all tissues and organs in the body. This information is present mainly in the form of antigens and proteins, usually present in very low concentrations. Therefore, blood sampling and analysis are of prime interest for both medical and science applications, and hold a central role in the diagnosis of many physiologic and pathologic conditions.

Current blood tests require expensive and non-easy to use instruments. It is very common for nurses and laboratory personal to be trained on the use of these instruments and on how to interpret the results. The duration of the test depends on the type of assay. But there are situations where the time-to-result and accuracy are critical to determine the condition of a patient such in a surgical procedure. In these situations, a point-of-care device would be beneficial. Also, blood tests require withdrawing milliliters of blood from the patient, which make the process inconvenient and sometimes painful for very young patients. In some tests, blood is combined with several reagents which can be expensive, so it's desirable to reduce the volume of blood in such cases.

Blood cells typically interfere with assays based on optical measurements and can reduce the sensitivity of biochemical assays (e.g. ELISA). So extraction of pure plasma from blood is a common preparation step for blood analysis. In most cases, the centrifuge is used to perform this step. The purified plasma is used for the subsequent assays. However, these processes involve multiple handling steps that are prone to introduce errors because samples can be misplaced or mis-labelled. There is a need to reduce the number of manipulation steps as wells the length of the assay. Automation and integration of these in a single unit is a highly desirable characteristic. Furthermore an arrangement which allowed for separation of the sample with very low shear stress so the blood cells are not lysed and the platelets are not activated would be highly desirable.

There is therefore a continued need for devices and methodologies for fabricating such devices which overcomes these and other problems.

### SUMMARY

These and other problems are addressed by a microfluidic device in accordance with the present teaching which provides for the filtering of whole blood samples to separate the plasma component from the rest of a blood fluid sample. The device desirably incorporates a step within a fluid path that serves to vary the height of the fluid path immediately prior to an assay region. In this way the blood cells within the fluid sample will be biased through sedimentation out of the fluid flow. The step could serve to reduce the height of the fluid path or could also be arranged to increase the depth of the fluid path—by formation of a trench. Such a latter arrangement may require a first and second step to generate the side walls of the trench. Fluid flow within the device is achieved using a degassing principle.

The invention therefore provides a device according to claim 1 with advantageous embodiments being detailed in the dependent claims. Independent claims directed to a blood assay system and a device are also provided. The invention also provides independent method claims with advantageous embodiments provided in the dependent claims thereto. These and other features of the invention will now be described with reference to exemplary embodiments of the invention.

### BRIEF DESCRIPTION OF THE DRAWINGS

FIG. 1A shows in schematic form a section through a channel of a microfluidic device in accordance with the present teaching incorporating a step within the fluid path to effect a separation of blood cells from plasma within a whole blood fluid sample.

FIG. 1B shows a modification to the arrangement of FIG. 1A where a first and second step are used to fabricate a trench within the fluid path, the trench providing for the passage of platelet-rich plasma leaving the trench region while the rest of blood cells are captured in the trench.

FIG. 2A shows a schematic time flow sequence for a fluid advancing towards the step of FIG. 1A and how the blood cells are filtered by the step.

FIG. 2B shows an equivalent schematic time flow sequence for the arrangement of FIG. 1.

FIG. 3 shows in schematic plan form how a plurality of channels may be provided in a multiplexed arrangement for simultaneous testing of multiple samples.

FIG. 4 shows how filling times for the channels are affected by the initial degassing time.

FIG. 5 shows results from use of a device in accordance with the present teaching for detection of biotin in whole blood.

FIG. 6 shows intensity plots for measurement of the biotin of FIG. 5 whereby avidin is immobilized in the assay region.

FIG. 7 shows an arrangement whereby blood and reagents can be combined to form a gradient.

FIG. 8 shows an example of how a mixing of blood and a reagent may be effected.

### DETAILED DESCRIPTION OF THE DRAWINGS

The teaching of the present invention will now be described with reference to an exemplary arrangement whereby a microfluidic device comprises a channel having a first region for separation of a whole blood sample and a second region where a plasma component of that sample may be analysed. Within the context of the present teaching it will be understood and appreciated by the person skilled in the art that these exemplary arrangements are provided to assist in an understanding of the teaching of the present invention and it is not intended to limit the teaching to such exemplary arrangements as modifications can be made without departing from the scope of the present teaching.

In accordance with this exemplary arrangement, a microfluidic device is provided which enables a separation of a fluid into its constituents based on a sedimentation principle. Such a separation or filtering allows for a less dense constituent of the fluid to pass downstream of a filter region within the fluid path where it may be subsequently captured for analysis purposes. The capture is effected through the affinity of surface bound capture molecules or target sites for the predetermined constituent of the fluid sample. Once bound into a sandwich assay, subsequent analysis provides information on the nature of the initial fluid sample. Within the present context an exemplary fluid, whole blood, will be described for the purposes of understanding the teaching of the present invention.

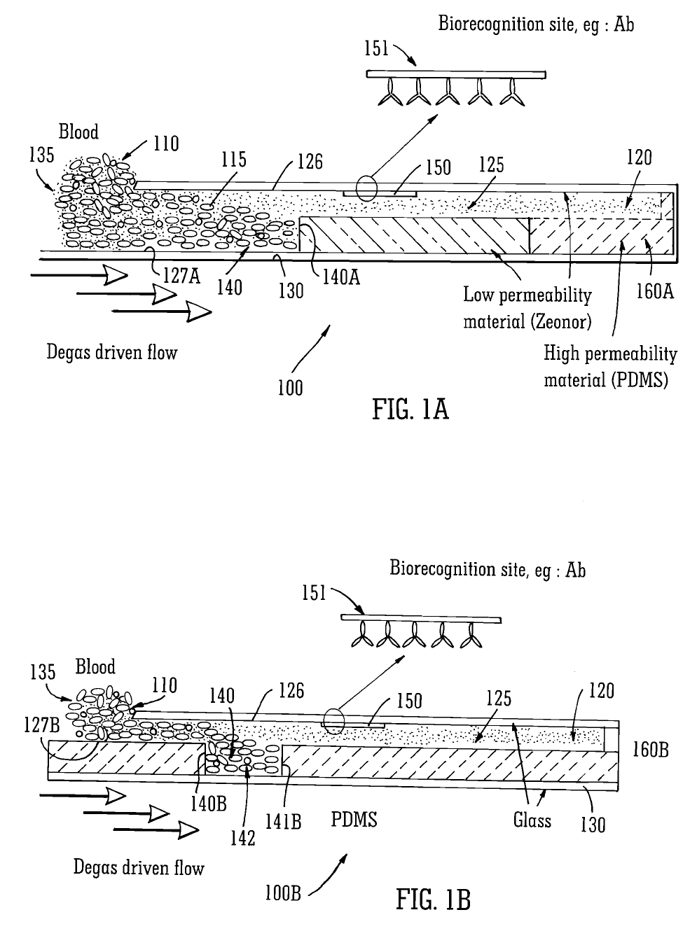

As shown in FIGS. 1A and 1B where the suffixes A and B refer to the same item in each of FIGS. 1A and 1B respectively, a microfluidic assay device 100 for assaying a fluid having first and second constituents is provided. In this example the fluid 110 is blood having a first constituent, the blood cells 115 with a density greater than the second constituent—the blood plasma 120. The device comprises a fluid path 125 defined within a substrate 130. The fluid path defines a closed channel and has an inlet 135 for allowing introduction of the fluid into the fluid path 125. By a closed channel is inferred a channel having side walls, a roof 126 and a floor 127.

While the fluid path represents a continuous route through the device for the fluid introduced through the inlet 135, it may be considered as comprising a number of distinct regions. Firstly a filter region 140 is provided downstream of the inlet 135. The filter region 140 provides for the capture of more dense particles or constituents within the fluid flowing through the fluid path. In the arrangement of FIG. 1A, the filter region comprises at least one step 140A defined in a surface of the fluid path—FIG. 1A shows the step 140A being upstanding from the floor of the fluid path. The step 140A defines a vertical surface in a direction substantially perpendicular to the direction of fluid flow through the fluid path. By providing such a step, the blood cell constituents of the fluid sample will sediment out of the fluid sample and preferentially collect in the filter region—thus providing a sedimentary collection of particles of the first constituent within the filter region.

In the arrangement of FIG. 1B, the filter region 140B comprises a first 140B and second 141B step arranged relative to one another to define a trench 142 within the fluid path. The trench provides for a lowering of the channel floor in the region of the trench and as a result of the gravitational effect on the particles within the fluid, those heavier particles, the blood cells, will tend downwardly into the trench where they are captured- effecting a sedimentary collection of particles of the first constituent within the filter region.

In both the arrangement of FIG. 1A and FIG. 1B a step provided in the surface of the fluid path effects a filtering of more dense constituents of the fluid sample so that they are taken out of the fluid flowing within the fluid path. The filtering of these more dense constituents of the fluid means that the fluid that passes onwardly within the fluid path downstream of the filter region 140 comprises substantially only the less dense constituents of the original fluid, in this exemplary arrangement the plasma.

To specifically target constituents of the plasma, an assay region 150 downstream of the filter region 140 is provided. The assay region in each of these examples comprises a plurality of biorecognition sites 151. By suitable patterning of the assay region it is possible to provide a multiplexed biorecognition matrix. Each of the bio-recognition sites are configured or selected for specific targeting of particular particles within the plasma. Within the present specification the term particle is intended to be broadly construed to include proteins, agents or antigens and other molecules or compounds as appropriate which are present within the plasma or other fluid and can be targeted for capture within the assay region. These bio-recognition sites represent capture agents or component thereof and are selected so as to be attachable to a surface of the assay region and having a binding affinity for the particles of the second constituent. By suitably treating or otherwise providing surfaces on which functional groups may be provided it is possible to covalently immobilize bio-molecules or other custom designed functionalities. The specifics of the capture elements will depend on the nature of the assay to be conducted but as will be appreciated by those of ordinary skill such capture elements may typically comprise one or more of antibodies, DNA, aptamers, recombinant antibodies, proteins, protein fragments, peptides etc. In targeting specific particles, which will then preferentially bind to the capture agents a sandwich assay arrangement may be provided within the assay region. This sandwich assay may then be analysed using a variety of techniques such as for example a luminescence response of the assay to excitation, or with different analytical techniques, for example surface and liquid techniques, such as Surface Plasmon Resonance based analysis, ellipsometry, electrical or optical measurements. It will be appreciated that certain of these techniques require specific treatment of the surfaces of the fluid path within the assay region such as for example a coating or patterning of one or more of the surfaces with a metallic layer, such as for example gold, silver, or other suitable materials.

In the exemplary arrangements of FIG. 1, the step defines a boundary between the filter and assay region. In both arrangements, i.e. FIG. 1A and FIG. 1B, a top surface 126 of the fluid path, the roof of the fluid channel, defines a planar continuum, i.e. continues uninterrupted through this boundary. As such while the step is shown as being defined in the floor 127 or bottom surface of the fluid path in exemplary arrangements, the step may be defined in any surface other than the top surface.

It will be appreciated from a visual inspection of FIG. 1 that in these exemplary arrangements the height of the fluid path within the assay region is less than the height of the fluid path within the filter region. While it is not intended to be limited to any one set of exemplary parameters typically the height of the fluid path within the assay region is less than about 10% of the height of the fluid path within the filter region. The height of the assay region is predetermined so as to control a flowing of the fluid within the fluid path from the filter region to the assay region and is selected to allow binding of particles of the second constituent to the capture agent and reduce background signal so as to form a detectable target capture agent binding complex. The main parameters that affect the ideal binding of particles and reduction of background signal are the antigen diffusion coefficient and the lateral flow velocity which will determine the antigen travel velocity and the interaction time with the surface, the capture agent (antibody) binding coefficients which will determine the probability of effective capture, the label and or antigen concentrations, non-specific binding and the detection sensitivity which will determine the amount of background noise that has to be reduced by tuning the height of the detection channel region. Using one or more of these parameters it will be understood by the person or ordinary skill that it is possible to determine an appropriate height and/or length for the assay region to ensure sufficient capture for analysis purposes.

Desirably, surfaces of the assay region are fabricated from a high gas-diffusibility coefficient material so as to allow for effective bubble-free priming of fluid through the detection or assay region and eliminate back pressure problems that can affect the device sealing. It will be understood from an examination of FIG. 1B that the surfaces are fabricated from PDMS whereas in the arrangement of FIG. 1A, the surfaces are low permeability Zeonor™. It will be understood that each material has advantages and disadvantages and the ultimate selection of an appropriate material will be determined on the basis of the device application. For example by using a detection region with a high permeability material there are associated advantages of bubble free self priming and reduced back pressure requirements. However such a material suffers in providing less even flow rates as the pumping effect of the low permeability material changes with the % fill of fluid. In contrast by providing the assay or detection region with surfaces fabricated from a low permeability material the advantages include a more regular or even flow rate as the pumping effect of the low permeability material does NOT change with the % fill of fluid. A disadvantage is that bubble free self priming is an issue, with a requirement for back pressure.

To allow for the propagation of a fluid introduced through the inlet 135, the device further includes or comprises a propulsion region 160 in fluid communication with the fluid path. The propulsion region comprises an exposed surface of a high permeability material such as polydimethylsiloxane (PDMS). It will be appreciated that PDMS is an example of a silicon based organic polymer which provides a hydrophobic surface which promotes nonspecific adsorption or even absorption of small molecules and gasses into the bulk mass of the substrate. In accordance with the present teaching prior to introduction of the fluid into the fluid path, the device is provided within an evacuated chamber such that an evacuation of the pores within the high permeability material is effected. By evacuating these pores, on subsequent introduction of a fluid into the fluid path a pressure differential is generated across the filter region through absorption of gases within the fluid path so as to effect a flow of the fluid through the fluid path. This will be discussed in more detail later but effectively the propagation process of the fluid within the fluid path is a passive arrangement requiring no active pumping or the like but rather acts due to a degassing principle whereby gases within the fluid path are absorbed into the porous material. This creates a reduction in the pressure within the fluid path in advance of the fluid, such that the fluid is drawn along the fluid path further into the device structure.

In the example of FIG. 1A, the propulsion region is located downstream of the assay region, the surface of the assay region being fabricated from a low permeability material such as Zeonor™ which will be appreciated is an example of a cyclo olefin polymer (Cop) engineering thermoplastic. This demarcation between the propulsion region and the assay region is advantageous for reasons including the flexibility of using convenient materials for structuring most of the device. For example it can be convenient for mass fabrication to use polymers that are compatible with injection moulding and that are very low cost. Furthermore the demarcation between the propulsion region and the assay region is advantageous in that the flow rates can be kept more constant because the propulsion rate will not change with the filling of the device.

In the arrangement of FIG. 1B, high permeability surfaces formed from the exemplary PDMS material extend throughout the entire fluid path such that the exact location of the propulsion region relative to the filter and/or assay regions is indeterminate. In this arrangement diffusion of the gases within the fluid path into the PDMS bulk material occurs throughout the entire length of the fluid path or channel. However as long as there is a sufficient volume of available material which may absorb these gases so as to create the pressure differential along the fluid path, the fluid will continue to propagate along the fluid path.

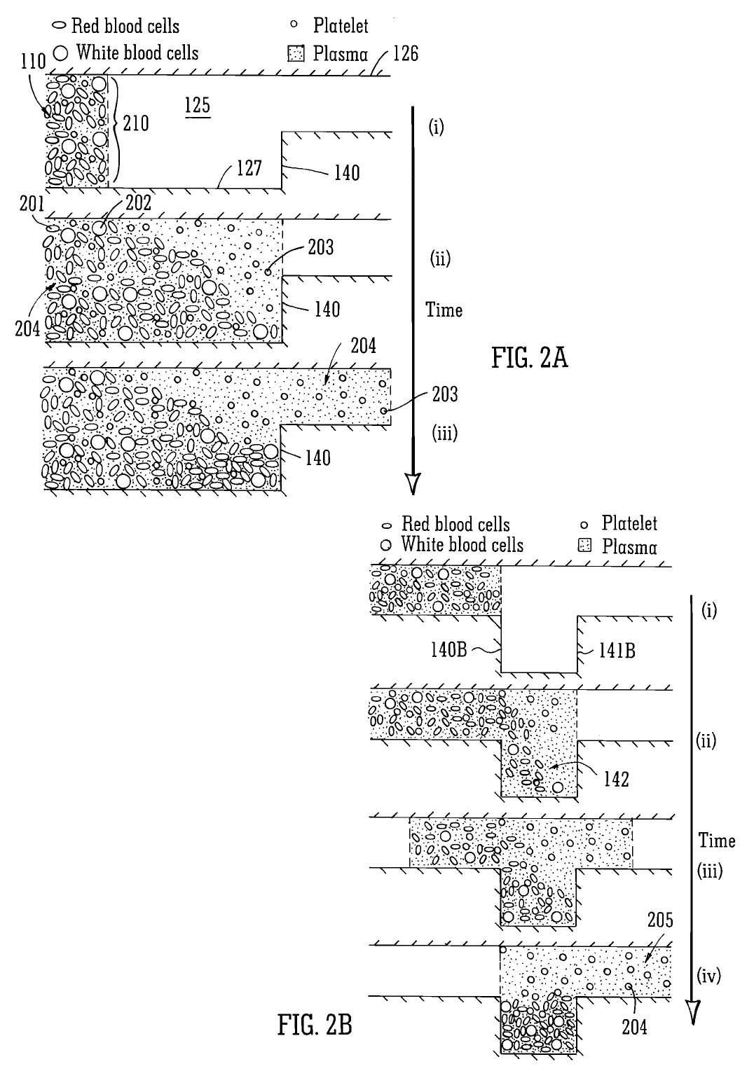

FIG. 2A shows in more detail a propagation of an introduced fluid 110 within the fluid path 125 of the arrangement of FIG. 1A. In this arrangement the fluid is the exemplary whole blood sample that comprises red blood cells 201, white blood cells 202, platelet 203 all suspended within a plasma constituent 204. When introduced into the fluid path through the inlet—shown in FIG. 1A, the fluid will extend across the entire channel to create a fluid front 210. This extends from the top 126 to the bottom 127 of the fluid path 125. As the path is a closed channel, only open via the inlet, the filling of the path by the fluid means that any air or other gases located within the channel forwardly of the fluid front 210 are effectively trapped within the fluid path. In the event that the surfaces of the channel were not porous and evacuated, this would result in an increase in pressure to the front of the fluid, resulting in a force acting against the movement of the fluid into the channel. However in accordance with the teaching of the present invention, one or more porous evacuated surfaces are provided forwardly of the advancing fluid 110. The increase in pressure resultant from the movement of the fluid into and along the channel causes am increase in pressure of the defined volume of gases located in the fluid path. Once the pressure is sufficient for the gases to migrate across the surface boundary from the fluid path into the pores of the bulk medium defining the surfaces of the channel, there is a resultant reduction of pressure within the fluid path which allows the fluid to advance forwardly again.

As is shown in the sequences i), ii) and iii) of FIG. 2A, this is a dynamic process that allows the fluid to move forwardly into the fluid path 125. In accordance with sedimentation principles, more dense constituents of the fluid will tend to migrate, under the influence of gravity, towards the bottom 127 of the fluid path 125. On encountering a barrier, in this instance the step 140, the more dense constituents—the white and red blood cells—are trapped and do not continue further into the device. The platelets 203 suspended within the plasma 204 continue along the path into the assay region of the device.

In an exemplary arrangement whereby the separation is used to separate the blood cells from the plasma, for the separation to function within the step, the white and red cells' residence times within the deep area (the channel area before the step 140) should be sufficient to allow them to sediment below and not enter into the assay region defined after the step 140. It will be appreciated that in exemplary arrangements such as those that may be fabricated in microfluidic devices, the height of the assay region will be about 80-μm, the height of the deep area before the step 500-μm and the height of the step 140, 420-μm.

Since blood cells are generally 10% more dense than the surrounding plasma they will sediment at a rate of 50-110 μm/min. As long as the whole blood flow velocity into the step separator channel is such that the blood cell residence time in the deep area is in the order of minutes, the white and red blood cells will sediment below the critical height defined by the step 140, such that the fluid flow field will not be able to displace them into the shallow channel area.

This will effectively capture the cells at the bottom of the deep area through a sedimentation process and separate them from the blood plasma that would continue to flow into the shallow channel area. The residence time can be controlled by the size of the deep area and the degassing driven flow rate.

FIG. 2B shows an equivalent process for the filling of the trench 142 of FIG. 1B. In this arrangement, it is similarly, the filling of the evacuated pores of the bulk substrate by the gases provided forwardly of the proceeding fluid that causes a reduction in pressure in the non-filled volume of the fluid path to the front of the fluid. This reduction in pressure induces motion of the fluid forwardly so as to equalise a pressure. As long as the available volume of pores within the substrate is sufficient to accommodate the displaced gases from the fluid path, the fluid will continue to propagate or move forwardly within the fluid path. In the arrangement of FIG. 2B, it is a lowering of the bottom surface of the fluid path by provision of the first 140B and second 141B steps to define the trap or trench 142 that serves to filter the heavier white and red blood cells from the plasma suspension. Subsequent to the trench, i.e. downstream of the filter region of the fluid path, the fluid is predominately a platelet enriched plasma. It is this filtering that effects the separation of the blood sample and it is the filtered or separated sample that then enter the assay region where capture elements selectively target constituents of the filtered sample.

The microfluidic device described heretofore has been representative of a single fluid path or channel structure having one inlet and one channel or path having filter and assay regions. Using the present teaching it is possible to multiplex such elements so as to have a plurality of channels located within the same substrate. These individual channels could be fabricated so as to share common inlets or propulsion regions or could also be simply fabricated as a plurality of channels each with independent inlets, fluid paths an/or propulsion regions as required. An advantage of providing a plurality of fluid paths is that a plurality of the same assays may be conducted for statistical purposes and/or a plurality of alternative tests may be conducted on the same sample in parallel.

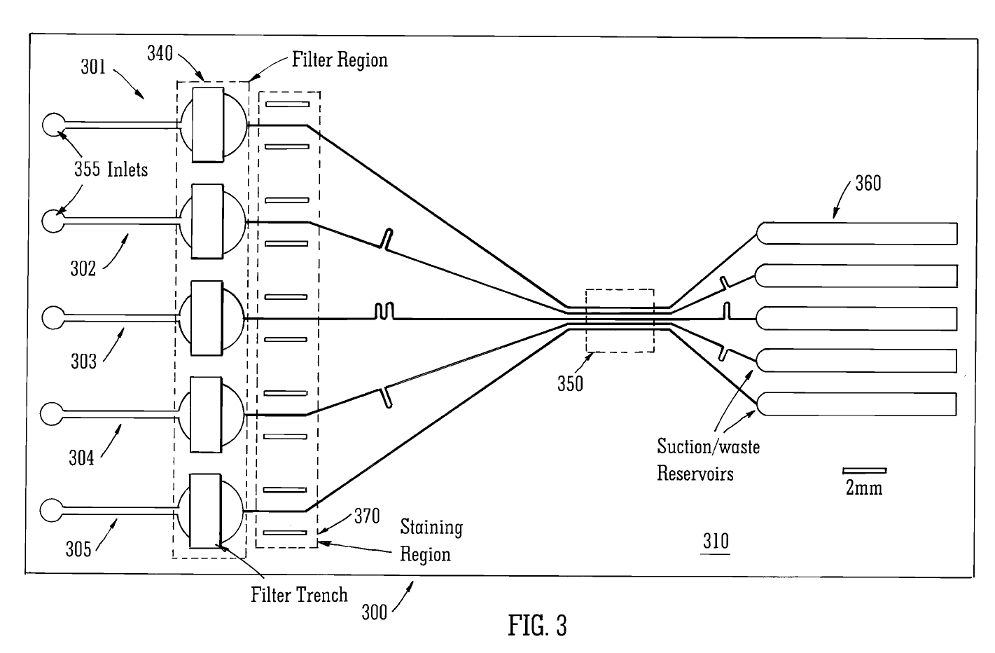

FIG. 3 shows an example of an integrated and multiplexed device 300. This exemplary device 300 consists of 5 sample processing pathways 301, 302, 303, 304, 305. The pathways of this arrangement are each provided on a common substrate 310, and in this exemplary arrangement share common dimensions. It will be understood however that one or more of the geometries of individual pathways could differ from others. The arrangement of FIG. 3 is based on a similar structure to that previously described in FIGS. 1B and 2B, but it will be understood that this is an exemplary arrangement of the structures that could be employed. Each fluid path starts at an inlet 335, which is upstream of a fluid filter region 340, as provided in this arrangement by a trench within the fluid path. On separation of the platelets from the white and red blood cells, the fluid passes by a staining region 370 which contains staining anti-bodies. Such a region was not described previously but will be understood as being useful in tests which require a precursor treatment of the fluid prior to the assay region so as to enable capturing of the desired constituents by the capture elements.

Examples of such staining reagents include fluorescent detection antibodies, which could be stored in a dried form between the trench and the functionalized bio-recognition area within the assay region. The purified or filtered plasma flows through the staining region where dried fluorescently labelled anti-bodies are dissolved and mixed into blood plasma. The channel then continues through to the assay region 350 where several capture antibodies are immobilised on one or more surfaces of the channel.

Within the assay region, the reagent would mix with the immobilized antibodies within the assay region. Non-bound fluorescent components are then washed out by the continuing plasma flow, such that subsequent excitation of the assay region and detection of the resultant luminescence would have minimal contribution from luminescent sources within the bulk media of the fluid.

In the arrangement of FIGS. 1 and 2, the capture agents are provided a priori to introduction of the fluid into the device. One useful technique to provide such agents is by use of microcontact printing. Using such a technique, once a capture agent is provided or coated onto the surface of the channel it will remain non-used and typically dry within the device until use of the device.

Each of the fluid paths in this arrangement are coupled to a dedicated propulsion region 360, which generates the impetus for movement of the fluid through the channels but also provides a waste region for collection of surplus sample that has passed through the entire length of the channel. Using devices having the dimensions which are shown in FIG. 3 (trench depth 1-mm, channel depth 80-μm and the smallest channel with 50-μm, provide for whole blood analysis using volumes of the order of 100 nL.

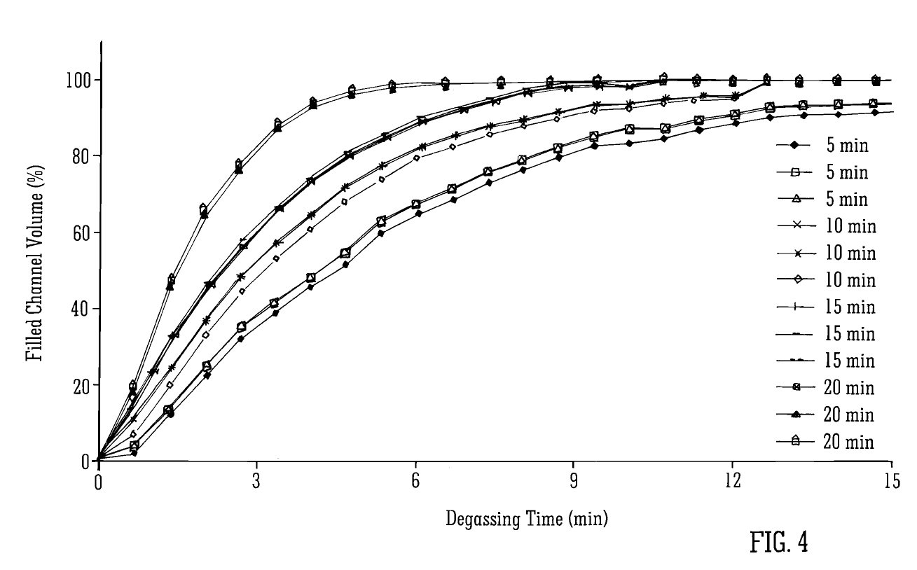

It will be appreciated that propulsion of the fluid through the closed channels of the device is achieved through a degassing methodology. As has been described with reference to FIGS. 1 and 2, by providing surfaces of the fluid paths in a high permeability material such as PDMS it is possible to take advantage of a flow phenomenon that is produced within such closed PDMS channels that have been degassed within a dessicator (under vacuum conditions) for several minutes. After degassing, PDMS begins to absorb air, so a closed air filled channel would begin to have a lower pressure. This generates a pressure difference between the outside of the chip and the closed channel. This pressure difference then drives the fluid into the channel. The fluid filling rate of the channel is dependent of the degassing time. FIG. 4 shows an example of how the available flow times can be modified by suitable immersion within the degassing chamber. For example using a 10 min degassing time a flow rate of rate 10 nL/min can be achieved, so that a sample of 100 nL could be fully introduced into the fluid path within 10 minutes.

It will be understood that for an immune assay to function properly, it is preferable to have a defined area within the channel wherein the capture anti-body is located. In arrangements described heretofore this may be achieved through a patterning and bonding of the capture anti-body to one or more surfaces through techniques such as micro contact printing, channel flow-through, direct printing, etc. By fabricating the device in a multi-layer manner it is possible to define a fluid path within a first device substrate and then use a second device substrate located on top of the first to effect provision of a roof or ceiling to close the channel. For example by fabricating the ceiling of the device using a glass substrate glass based immobilization chemistry may be used to pattern individual bars using micro contact printing and physisorption. This, in an exemplary arrangement may provide for the patterning of individual strips of 15 μm wide streptavidin-biotin assay bars were patterned transverse to the fluid path such that a plurality of individual bars were provided across the fluid path. By selective patterning individual ones of the bars could be modified or configured for selective targeting of specific analytes within the sample. This therefore may be used to generate target sites for individual plasma based proteins. By providing a device such as shown in FIG. 3 each of the individual channels may be used for different concentration samples to provide a simultaneous analysis of a plurality of samples within the same conditions.

It will be understood that use of such a multi-layer structure also allows the patterning of the assay region prior to the formation of the ultimate channel by judicious patterning and then placement of the second device substrate on the first device substrate. This will result in the capture antibodies being located on the ceiling or upper surface only of the channel. By using a non-permanent assembly mechanism, on effecting capture of the relevant analyte within the capture region, it is possible to then disassemble the device to allow for removal of the second device substrate with the capture analyte provided thereon. If this is provided from optically transparent materials such as glass or the like it may then be introduced directly into an optical analysis arrangement such as for example a standard microarray scanner.

As was described herein to generate the necessary induced fluid flow through the closed fluid path, at least a portion of the surface of the fluid channel needs to be fabricated from polydimethylsiloxane (PDMS) or materials with similar properties and air absorption rates (gas permeability and solubility). This exposed bulk material may be provided in a distinct region of the fluid path or may partially overlap with for example the assay and/ or filter regions. To preserve the degassing flow capabilities the device can be long-term stored within a vacuum package. The assembly of the device does not require an irreversible (permanent) bonding between the glass substrate and the polymeric structure which facilitates its fabrication, manufacturability and operation. Also a combination of hydrophilic and hydrophobic polymers could be used for driving the fluid flow. In this way, other polymers other than PDMS or PDMS with special treatment can be used so that a hydrophilic surface is generated and capillary flow can be used.

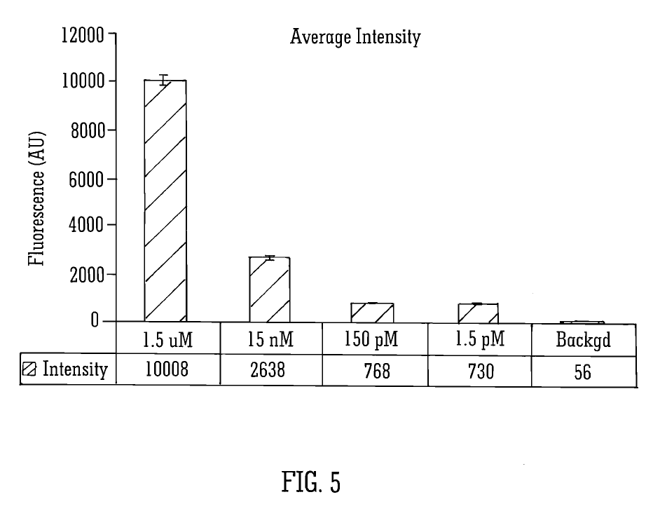

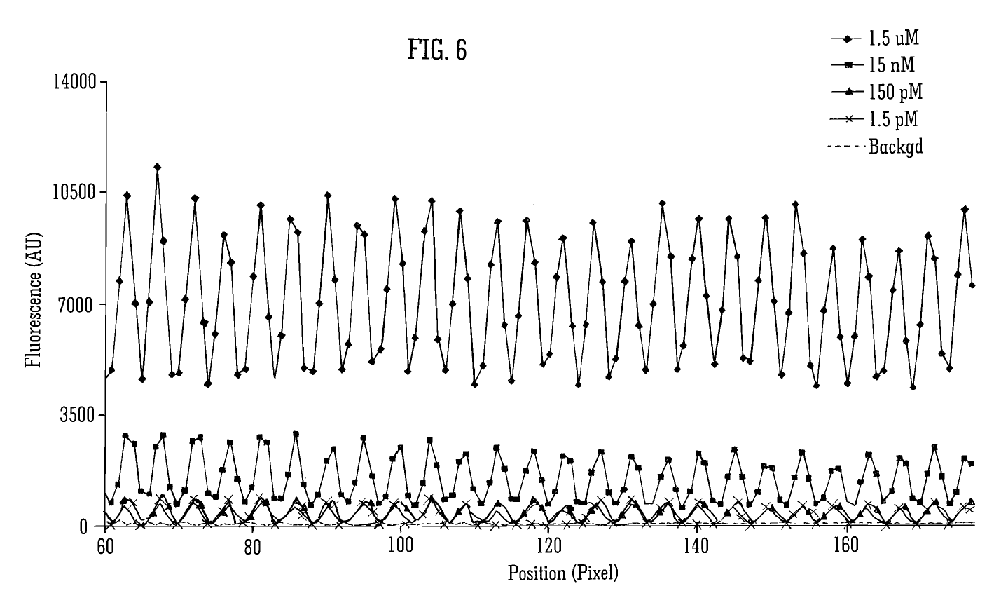

Once the plasma has been driven passed the assay region so as to effect a capture of predetermined target constituents of the plasma, it is possible to analyse the assay region. This could be done contemporaneously with the flow of the sample through the device or could be done subsequently and at a different location. By using optically transparent substrates it is possible to conduct in situ luminescence experiments where the assay region may be excited and the resultant luminescence captured. FIGS. 5 and 6 show examples of such analysis whereby an avidin and biotin system provide a level of detection of 1.5 pM of biotin in whole blood, and the reproducibility relative standard error is 13.6%.

It will be appreciated that as a device in accordance with the present teaching defines a closed system, i.e. it has inlets but no outlets, the total volume used in the system can be controlled by setting the volume of the suction chambers 360 or dead-end channels. Once the suction chambers are completely filled the flow stops across the entire chip. With no driving forces, the flow over the trench or step also ceases and thus cells already captured in the trench will remain there, without overflowing into the biomarker recognition area 350. In this way the volume that may be introduced and which passes down into the filter region may be self limited by the nature and dimensions of the propulsion region, i.e. once the evacuated material is fully saturated it no longer effects a degassing based propulsion and no more sample would be drawn towards the step or trench.

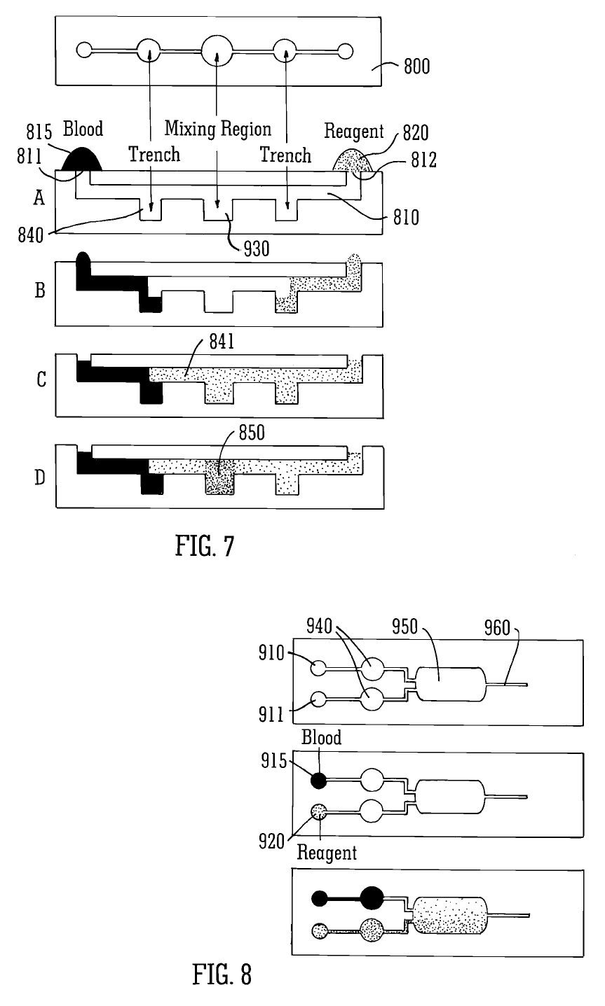

When the reagents cannot be immobilized inside the microfluidic device and only wet reagents can be used, for example when very fragile antibodies or proteins have to be used or when it is desired to provide an in-solution reaction, the device offers the versatility to configure it in different ways to allow for the usage of liquid reagents. FIG. 7 shows a modification to the devices described heretofore wherein blood plasma is combined with a liquid reagent, each being introduced at different ends of a fluid path. In this way the device provides for a mixing of the two fluids. In this exemplary arrangement of mixing of the sample with a suitable fluid reagent a sample pathway 810 is provided having first 811 and second inlets 2. In this example, the first and second inlets are provided at opposite ends of the pathway. A blood sample 815 is introduced through the first inlet 811 and a reagent sample 820 is provided through the second inlet 812. A mixing region 830 is provided between the first and second inlets and the fluid pathway between each of the inlets and the mixing region is configured according to the nature of the fluids introduced to ensure that each of the fluids introduced at the two inlets will meet at the mixing region 830. As it is important that the blood sample progresses past a filter region 840, in this exemplary arrangement provided by a trench in the fluid pathway, so as to achieve the necessary separation of the blood cells from the plasma 841, and as this will affect the time taken for the blood to travel to the mixing region, one exemplary arrangement for ensuring that the two fluids meet concurrently within the mixing region is to provide the fluid pathway between the second inlet and the mixing region as a mirror image of the fluid pathway between the first inlet and the mixing region. Of course other designs or configuration could be used incorporating for example meander patterns, greater dimension channels or the like. Indeed if separation of the blood sample is not required, then the filter region could be omitted. By having the samples meeting a centralised mixing region and each of the fluids subsequently blocking the passage of their opposing fluid through the mixing region to the other side, it is possible to achieve an efficient mixing of the sample and reagent for analysis. A readout signal 850 may be determined by suitable analysis of the mixing region—which in this configuration is analogous to the assay region of the previous exemplary arrangements described herein.

In operation, a drop of blood and a drop of reagent are loaded into the left and right inlet, respectively. Blood starts flowing into the channel and eventually only plasma overflows from the trench as described previously. The reagent also starts flowing at the same time from the right side. To allow an even mixing of both solutions in the mixing region, a trench is also added on the right side, so both solutions arrive at the same time in the mixing region. The mixing region can adopt different geometries to achieve a more efficient or tailored mixing.

Another modification to that described heretofore is described in FIG. 8 whereby a microfluidic device 900 configured to effect generation of a gradient of plasma and a reagent is provided. A first 910 and second 911 inlet are provided to dedicated channels that pass through a filter region 940 such as provided by a trench arrangement. A whole blood sample 915 and a reagent sample 920 are introduced into each of the first and second inlets. The plasma is filtered with the same principle previously described. Downstream of the filter region each of the first and second fluid paths combine into a single gradient chamber 950 where the plasma sample is combined with a drug or reagent from the second fluid pathway. Both plasma and the reagent enter in a chamber where they form a gradient. A common propulsion region 960 is provided downstream of the gradient chamber 950 so as to provide the necessary impetus through the degassing methodology previously described to effect movement of the two fluids through the closed channels. Such gradient chambers could be used for a variety of purposes or applications including for example to stimulate cells or samples with different concentrations of a reagents eg. drug, or stain, or transcription factor, etc. and see the effect of this. This will be appreciated is an alternative example of use of a device in accordance with the present teaching to the blood antigen detection assay described heretofore

While it is not intended to limit the present teaching to any one specific manufacturing technique the following describes an exemplary arrangement for fabricating a device in accordance with the present teaching.

The microfluidic channels were fabricated using standard soft lithography replica molding techniques. Briefly, a mould was created through a single-layer process using negative photoresist, SU8-2100 (Microchem U.S.A.), was spun onto a clean silicon wafer using a spin-coater (P6700 Specialty Coating Systems, Inc., U.S.A.) to form a 80-μm thick layer. The photoresist was poured onto the wafer at 500 rpm, the angular speed was then ramped up to 2500 rpm for 30 sec with an acceleration of 300 rpm/s. Next, the wafer was soft-baked at 65° C. for 5 min and 95° C. for 30 min, followed by UV-exposure for 10 s at 9.5 mW/cm2 using a mask aligner (Karl-Süss KSM MJB-55W). The wafer was then baked for 5 min at 65 ° C. and 12 min at 95 ° C., and allowed to cool down to room temperature. Finally, the wafer was developed in SU8 developer (Microposit EC Solvent, Chestech Ltd., UK) for 4 min, rinsed with isopropanol, and N2 blown-dry.

PDMS (Sylgard 184, Dow Corning) was prepared with a 10:1 mass ratio (base to cross-linker); degassed in a vacuum chamber for 30 min; then poured on the SU8 mold to a thickness of ˜2 mm; and cured in a oven at 60° C. for at least 10 h. The PDMS was then carefully peeled off the mould. The PDMS was punched with a 2-mm outer diameter flat-tip needle (Technical Innovations, Inc, Tex., USA) to form the filter circular trenches. Up to 5 trenches were punched in one chip. The PDMS fluidic layer was placed in conformal contact with the glass slides, providing reversible sealing.

Prior to assembling the device the bio-recognition site on the top glass slide was patterned by microcontact printing to create 15 μm wide stripes of avidin (Sigma Adrich, U.S.A.) Patterned PDMS stamps were fabricated by pouring a 10:1 (v/v) mixture of Sylgard 184 elastomer and curing agent over a patterned silicon master. Fabrication of the patterned silicon master was done as follows: MICROPOSITTM S1818™ Positive Photoresist was spun at 5500 rpm for 30 sec on a silicon wafer. The coated wafer was then cured for 1 min on a vacuum hot plate at 115° C. UV light irradiated the photoresist layer for 20 sec through a photomask (Photronics, Mid Glamorgan, South Wales, UK). Resultant features were developed by dipping the master in developer MF319 (Chestech Ltd, Warwickshire, UK) for 40 sec and finally rinsed with water and dryed under nitrogen. Subsequently, masters were exposed to a vapour of (tridecafluoro-1,1,2,2-tetrahydrooctyl)-1-trichlorosilane (Sigma Aldrich Inc., Ireland) under vacuum for 1 h to facilitate the release of the PDMS mold after curing.

The mixture was cured for one hour in an oven at 60° C., then carefully peeled away from the master and left in the oven for another 18 h at 60° C. to ensure complete curing. Prior to inking the stamps were oxidized by exposure to UV/ozone for 10 min. This process causes the stamp surface to become hydrophilic, which ensures homogeneous spreading of the ink (i.e. the protein solution). The stamps were freshly prepared no more than two days prior to use.

It will be understood that exemplary arrangements of a fluid separator that allows in this exemplary application for plasma filtering from whole blood has been described. Such a device is capable of achieving close to 100% capture efficiency of red and white blood cells with no clogging. By use of a filter region that operates through sedimentary principles it is possible to provide platelet-rich plasma from a whole blood sample into an assay or detection region where multiple immunoassays can be performed. The separation is done with very low shear stress so the blood cells are not lysed and the platelets are not activated. Subsequent to obtaining the plasma, different types of analysis can be performed on the plasma. As has been described herein the device is particularly well suited for use in luminescence based analysis but is equally compatible with different analytical techniques, surface and liquid techniques, such as Surface Plasmon Resonance based analysis, ellipsometry, electrical or optical measurements. The plasma can be analyzed either in pure form or in a combination with a reagent.

In accordance with the present teaching movement of a fluid through a microfluidic device may be effected using a degassing based fluid flow method. Such a technique is controllable and stable enough to perform the separation and assay. The device can be evacuated and then stored in a vacuum-sealed container to extend the duration of the effect. It will be understood that by judicious selection of the surface area and material used to form the propulsion region that it is possible to control the flow velocity of a fluid sample through the fluid paths. Furthermore the volume of the propulsion area will affect and can determine so as to allow for a metering of the total volume flown through the entire system. This eliminates the need for pumps, valves and other control mechanisms. The high diffusion coefficient material is mostly enclosed the within a non-permeable material such that all the suction happens directly into the propulsion-and-metering cavity. This way the material operates longer in the saturation zone and thus maintains a more stable flow which directly improves the assay reproducibility. The high diffusion coefficient material can be present only in the propulsion region allowing a more constant flow, and the freedom to manufacture the rest of the device from any other material (including cheap injection moulded polymers). In fabricating the device, reversible bonding may be used for device assembly. These allows the patterning of one the layers with reagents (antibodies, DNA, aptamers, recombinant antibodies, or proteins, protein fragments, peptides, etc), called the analyte capture layer, independently of the device manufacture. This layer can be patterned using different methods such as microcontact printing, spotting, flow channel, and alike. It will be further understood that reagents can be immobilized or lyophilized in one or more of the surfaces of the same microfluidic device. Wet reagents could be also stored using a combination of other storage systems such as laser or pneumatic valves.

It will be understood that by using reversible bonding, the same device can be dissembled to allow for a cleaning of the fluid pathways so as to allow for re-use.

Furthermore, the analyte capture layer or assay region can be used for analyte post analysis such as MALDI-MS, PCR, or other analysis techniques.

A device in accordance with the present teaching is also suitable for use in mixing a wet reagent with blood plasma. One of the many uses of such a mixing mechanism is for coagulation monitoring, which can also be combined with the previously mentioned analysis and measurement techniques (SPR, ellipsometry, electrical and optical analysis, and other similar techniques).

While a trench structure can be used to effect a filter region, it will be appreciated that such a filter region may advantageously only employ a step structure is required, which relative to a trench arrangement, is more efficient and faster in filtration due to the longer sedimentation path and the faster initiation of the sedimentation. After filtration the analysis is done in an assaying channel that contains the detection region and has an adjusted channel height so that the in-solution background signal can be minimized eliminating the need for a washing step and increasing the system sensitivity. The flow in the system is generated with a propulsion-and-metering cavity made of a high diffusion coefficient material with a surface area that controls the flow velocity and a cavity volume that meters the total volume flown through the entire system. This eliminates the need for pumps, valves and other control mechanisms. The high diffusion coefficient material is mostly enclosed within a non-permeable material such that all the suction happens directly into the propulsion-and-metering cavity. This way the material operates longer in the saturation zone and thus maintains a more stable flow which directly improves the assay reproducibility.

It will be appreciated that an exemplary arrangement of a self-powered integrated microfluidic blood analysis system has been described that efficiently extracts blood plasma from small (typically less than 5 μL) samples of whole blood and performs multiplexed sample-to answer assay with picomolar sensitivity without any external pumping mechanisms. Using such an arrangement it is possible to then perform optical measurement based assays with improved sensitivity by removal of the blood cells prior to the assay. Separation of plasma on microfluidic devices has previously required arrangements such as umbilical tubes for flow propulsion and control and use syringe pumps, compressed air, or external electropneumatic systems, rotating platforms or the like which result in the ultimate device manufacture and control operation more complex and expensive. In this exemplary arrangement it is possible to effect separation without external tubes or driving mechanisms using a self-priming degassing-driven flow technique that causes blood to flow spontaneously into air-filled dead-end channels without external pumping mechanisms. In this way whole blood samples of volumes smaller than 5 μL can be introduced directly from the finger. The sample, once introduced may be filtered using a step arrangement that sediments white and red blood cells to the bottom of the fluid path allowing only cell-free plasma (including platelets) to overflow and continue into the sensing area of the self-priming tubeless device. Subsequent analyte detection is possible using a dedicated assay region such as for example the described avidin-biotin assay. Within this region a pattern capture agents such as for example 15 μm bars of avidin may be immobilized. Whole-blood samples may be spiked with different concentrations of fluorescently-labelled biotin with the results showing that picomolar detection of analytes in whole-blood can be readily achieved. Fluorescent readout of the device is one exemplary technique that may be used for analysis purposes and may be done by disassembly of the device to remove a patterned upper surface and inserting that lid into a standard microarray scanner. By fabricating the device in a multi-layer construct which does not require irreversible bonding between the utilised PDMS and glass layers, it can be easily disassembled, giving the user direct access to the captured analytes. This is advantageous in applications where the captured analytes need to be further examined, for example with PCR, MS, or other techniques. For multi-analyte detection, each avidin or other capture element bar can be replaced with a different probe, allowing the sensing of up to several thousand analytes in each blood sample.

Therefore although the invention has been described with reference to exemplary illustrative embodiments it will be appreciated that specific components or configurations described with reference to one figure may equally be used where appropriate with the configuration of another figure. Any description of these examples of the implementation of the invention are not intended to limit the invention in any way as modifications or alterations can and may be made without departing from the spirit or scope of the invention. It will be understood that the invention is not to be limited in any way except as may be deemed necessary in the light of the appended claims.

Similarly, the words comprises/comprising when used in this specification are to specify the presence of stated features, integers, steps or components but does not preclude the presence or addition of one or more other features, integers, steps, components or groups thereof.

1. A microfluidic assay device for assaying a fluid having first and second constituents, the first constituent having a density greater than the second constituent, the device comprising a fluid path defined within a substrate, the fluid path comprising:
an inlet for allowing introduction of the fluid into the fluid path;
a filter region downstream of the inlet, the filter region comprising at least one step defined in a sidewall of the filter region, the at least one step operably effecting a sedimentary collection of particles of the first constituent within the filter region;
an assay region downstream of the filter region, the assay region being configured for targeted collection of particles of the second constituent;
the device further comprising a propulsion region in fluid communication with the fluid path, the propulsion region comprising an evacuated porous material which operably generates a pressure differential across the filter region through absorption of gases within the fluid path so as to effect a flow of the fluid through the fluid path.

2. The device of claim 1 wherein the step is defined in the substrate in a direction substantially perpendicular to the direction of fluid flow through the fluid path.

3. The device of claim 1 wherein the step effects a reduction in height of the fluid path.

4. The device of claim 1 comprising a second step, the first and the second step defining a trench within the fluid path.

5. The device of claim 1 wherein the fluid path comprises a top surface, the step being defined in a surface other than the top surface.

6. The device of claim 5 wherein the top surface defines a planar continuum between the filter region and the assay region.

7. The device of any preceding claim 1 wherein the height of the fluid path within the assay region is less than the height of the fluid path within the filter region.

8. The device of claim 7 wherein the height of the fluid path within the assay region is less than about 10% of the height of the fluid path within the filter region.

9. The device of any preceding claim 1 wherein the step defines a boundary between the filter region and the assay region.

10. The device of claim 1 wherein the assay region comprises at least one capture agent or component thereof, the at least one capture agent or component thereof being attached to a surface of the assay region and having a binding affinity for the particles of the second constituent.

11. The device of claim 10 wherein the height of the assay region is predetermined so as to operably control a flowing of the fluid within the fluid path from the filter region to the assay region and is selected to operably allow binding of particles of the second constituent to the capture agent and operably reduce background signal so as to form a detectable target capture agent binding complex.

12. The device of claim 10 wherein surfaces of the assay region are fabricated from a high gas-diffusibility coefficient material so as to allow for the passage of fluid through the region without bubbles or back-pressure effects affecting the flow.

13. The device of claim 10 wherein surfaces of the assay region are fabricated from a low gas-diffusibility coefficient material.

14. The device of claim 1 fabricated at least in part from polydimethylsiloxane (PDMS).

15. The device of claim 14 wherein surfaces of the fluid path in at least the propulsion region are exposed PDMS.

16. The device of claim 1 comprising a plurality of fluid paths.

17. The device of claim 16 wherein the plurality of fluid paths share a common propulsion region.

18. The device of claim 16 wherein the plurality of fluid paths share a common inlet.

19. The device of claim 10 wherein the capture element comprises one or more of antibodies, DNA, aptamers, recombinant antibodies, proteins, protein fragments, or peptides.

20. The device of claim 10 wherein the at least one capture agent or component thereof is attached to a ceiling of the fluid path.

21. The device of claim 10 wherein the at least one capture agent or component thereof is patterned on a surface of the assay region.

22. The device of claim 1 fabricated as a multi-layer structure.

23. The device of claim 22 wherein a first layer of the device is removable post collection of particles of the second constituent, the collected particles being provided on that layer to allow for a subsequent analysis of the collected particles.

24. The device of any preceding claim 1 wherein the fluid path comprises a second inlet, the second inlet operably allowing for introduction of a second fluid into the device, the fluid path defining a mixing region wherein the first and second fluids may mix.

25. A blood assay system comprising:
a microfluidic assay device for assaying a fluid having blood and plasma, the device comprising a fluid path defined within a substrate, the fluid path comprising;
an inlet for allowing introduction of the fluid into the fluid path;
a filter region downstream of the inlet, the filter region comprising at least one step defined in a sidewall of the filter region, the at least one step operably effecting a sedimentary collection of blood particles within the filter region;
an assay region downstream of the filter region, the assay region being configured for targeted collection of plasma;
the device further comprising a propulsion region in fluid communication with the fluid path, the propulsion region comprising an evacuated porous material which operably generates a pressure differential across the filter region through absorption of gases within the fluid path so as to effect a flow of the fluid through the fluid path, the first constituent being red blood cells and the second constituent plasma.

26. A microfluidic device for mixing a first fluid with a second fluid, the device comprising a fluid path defined within a substrate, the fluid path comprising:
a first inlet for allowing introduction of the first fluid into the fluid path;
a second inlet for allowing introduction of the second fluid into the fluid path;
a mixing region downstream of the first and second inlets, the mixing region providing for a combining of the first and second fluids within the fluid path;
the device further comprising a propulsion region in fluid communication with the fluid path, the propulsion region comprising an evacuated porous material which operably generates a pressure differential across the mixing region through absorption of gases within the fluid path so as to effect a flow of the fluids through the fluid path.

27. A method of fabricating a microfluidic assay device for use in assaying a fluid having first and second constituents, the first constituent having a density greater than the second constituent, the method comprising:
a. Defining a fluid path defined within a substrate, the fluid path comprising:
i. an inlet for allowing introduction of the fluid into the fluid path;
ii. a filter region downstream of the inlet, the filter region comprising at least one step defined in a sidewall of the filter region, the at least one step operably effecting a sedimentary collection of particles of the first constituent within the filter region;
iii. an assay region downstream of the filter region, the assay region being configured for targeted collection of particles of the second constituent;
b. defining a propulsion region in fluid communication with the fluid path, the propulsion region comprising an evacuated porous material which operably generates a pressure differential across the filter region through absorption of gases within the fluid path so as to effect a flow of the fluid through the fluid path.

28. The method of claim 27 comprising defining the step is defined in the substrate in a direction substantially perpendicular to the operable direction of fluid flow through the fluid path.

29. The method of claim 27 comprising providing at least one capture agent or component thereof within the assay region, the at least one capture agent or component thereof being attached to a surface of the assay region and having a binding affinity for the particles of the second constituent.

30. The method of claim 27 comprising defining a second step, the first and second step defining a trench within the fluid path.

31. The method of claim 27 wherein the fluid path comprises a top surface, the method comprising defining the step in a surface other than the top surface.

32. The method of claim 31 further comprising providing at least one capture agent or component thereof within the assay region, the at least one capture agent or component thereof being attached to a surface of the assay region and having a binding affinity for the particles of the second constituent and wherein the at least one capture agent on the top surface of the fluid path.

33. A method of separating and sampling a whole blood sample comprising:
providing an evacuated microfluidic closed channel;
introducing a blood sample into an inlet of the channel, the sample being induced to flow through the channel through a degassing mechanism within the channel;
filtering cellular matter from the sample through a sedimentary filter mechanism downstream of the inlet;
providing a bio-recognition sample area within the fluid path such that the filtered sample will pass through the bio-recognition area where particular constituents of the filtered sample may be captured for subsequent analysis.
